(19) United States
(12) Patent Application Publication
(10) Pub. No.: US 2021/0205061 A1
(43) Pub. Date: Jul. 8, 2021

(54) OCCLUSAL PRESSURE ANALYSIS PROGRAM

(71) Applicants: THE NIPPON DENTAL UNIVERSITY, Tokyo (JP); GC CORPORATION, Shizuoka (JP)

(72) Inventors: Hiroshi SHIGA, Tokyo (JP); Tomohisa SHIRAISHI, Tokyo (JP); Yukie NOGUCHI, Tokyo (JP)

(73) Assignees: THE NIPPON DENTAL UNIVERSITY, Tokyo (JP); GC CORPORATION, Shizuoka (JP)

(21) Appl. No.: 17/136,112

(22) Filed: Dec. 29, 2020

(30) Foreign Application Priority Data
Jan. 6, 2020 (JP) 2020-000370

(51) Int. Cl. A61C 19/05

(52) U.S. Cl. CPC A61C 19/05; A61C 2201/002

(57) ABSTRACT

Provided is an occlusal pressure analysis program capable of improving the accuracy of grasping the occlusal contact state in occlusal pressure acquisition by a pressure detection means, including a step of obtaining a pressure value for each minimum unit from which the pressure value can be recognized by a pressure detection means, and a step of, regarding each of the minimum units where the pressure value exists as a detection unit, separating the detection units into the detection units which can be regarded as due to occlusion and the detection units which are unclear whether they are due to occlusion, based on the pressure value.

### FIELD

The present invention relates to programs for analyzing occlusal pressure in an occlusal contact state of upper and lower tooth rows.

### BACKGROUND

Pressure sensitive films (pressure sensitive sheets) and sensor sheets are known as means for measuring occlusal pressure (for example, Patent Literatures 1 to 4). When a subject bites a pressure sensitive film, the pressure sensitive film is strongly pressed at an occlusal contact portion of the upper and lower tooth rows, and a visual or electrical change appears. Depending on the type of the pressure sensitive film, differences in shade or color may appear depending on the magnitude of the occlusal pressure (contact pressure). In this manner, the occlusal pressure of the upper and lower tooth rows can be known with the pressure sensitive film.

### CITATION LIST

### Patent Literature

Patent Literature 1: JP H06-213738 A

Patent Literature 2: JP H06-189980 A

Patent Literature 3: JP H06-180260 A

Patent Literature 4: JP 2005-279094 A

### SUMMARY

### Technical Problem

Pressure sensitive films show variations corresponding to the occlusal pressure at the site where the occlusal contact has occurred. This shows visual changes at the occlusal contact position, and the occlusal position and the pressure can be identified. However, since tooth surfaces have three-dimensional shapes with complicated irregularities, the pressure sensitive film experiences slippage while contacting the upper and lower teeth between the time when the subject starts to bite the pressure sensitive film and the lime when the subject reaches the final occlusal position, and the process of this slippage also appears as a visual change in the pressure sensitive film. The influence of complicated folding of the sheet and the influence of adhering matters at the time of analysis of the occlusal pressure also may appear.

Such variations and influences in the pressure sensitive film have sometimes disturbed accurate grasp of the occlusal contact state. That is, when the occlusal pressure is acquired with the pressure sensitive film, the occlusal pressure different from the occlusal pressure at the final occlusal state is detected in the occlusion process or the analysis process, which have sometimes disturbed accurate grasp of the occlusal contact state. This is not limited to pressure sensitive films, and the same thing happens with the means for electrically obtaining the occlusal position and the occlusal pressure.

An object of the present invention is to provide un occlusal pressure analysis program capable of improving the accuracy of grasping the occlusal contact state when an occlusal pressure is obtained from a pressure sensitive means.

### Solution to Problem

One aspect of the present invention is an occlusal pressure analysis program for obtaining an occlusal pressure distribution from a pressure sensitive means, the occlusal pressure analysis program including: a step of obtaining a pressure value for each minimum unit from which the pressure value can be recognized by the pressure sensitive means, and a step of, regarding each of the minimum units where the pressure value exists as a detection unit, separating the detection units into the detection units which can be regarded as due to occlusion and the detection units which are unclear whether they are due to occlusion, based on the pressure value.

Another aspect of the present invention is an occlusal pressure analysis program for obtaining an occlusal pressure distribution from a pressure sensitive means, the occlusal pressure analysis program including: a step of obtaining a pressure value for each minimum unit from which the pressure value can be recognized by the pressure sensitive means; a step of obtaining areas of detection groups, regarding the minimum unit where the pressure value exists as a detection unit and regarding a group of a plurality of the detection units that are existing continuously as each of the detection groups; a step of obtaining a pressure characteristic value of each detection group; and a step of separating the detection groups into the detection groups which can be regarded as due to occlusion and the detection groups which are unclear whether they are due to occlusion, based on the area of the detection group and the pressure characteristic value.

The occlusal pressure analysis program may include a step of separating the detection groups into the detection groups which can be regarded as due to occlusion and the detection groups which are unclear whether they are due to occlusion, by a threshold of the pressure characteristic value associated with the area of the detection group.

The occlusal pressure analysis program may be structured such that an upper limit of the pressure characteristic value at the threshold becomes smaller as the area of the detection group becomes larger.

The occlusal pressure analysis program may further include a step of separating the detection groups into the detection groups which are due to occlusion and the detection groups which are unclear whether they are due to occlusion, from at least one of the shape, distance from the other detection groups, und color of the detection group.

### Advantageous Effects of Invention

According to the present invention, it is possible to improve the accuracy of grasping the occlusal contact state in obtaining the occlusal pressure by the pressure sensitive means.

### BRIEF DESCRIPTION OF DRAWINGS

FIG. 1 is a diagram showing a flow of an occlusal pressure analysis program S10.

FIG. 2 is a diagram to explain detection units P and detection groups G.

FIG. 3 is a diagram schematically showing a distribution of the detection groups G.

FIG. 4 is a diagram to explain a structure of an electronic computer 10.

### DETAILED DESCRIPTION OF EMBODIMENTS

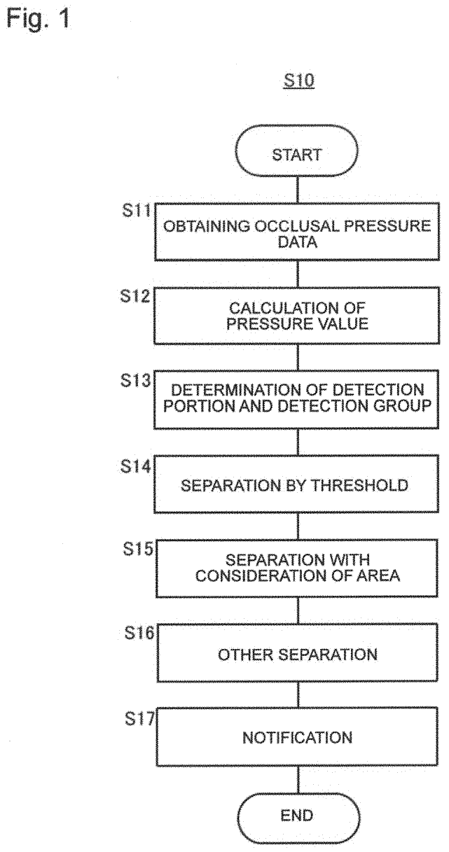

FIG. 1 is a diagram showing a flow of an occlusal pressure analysis program S10 according to one embodiment. As can be seen from FIG. 1, the occlusal pressure analysis program S10 includes steps S11 to S17. Hereinafter, each step will be described.

Step S11 of Obtaining Occlusal Pressure Data

In the step S11 of obtaining occlusal pressure data (it may be referred to as “step S11”), positional information and detection information are obtained with respect to a portion where a predetermined information is detected by the pressure sensitive means (in the case of a pressure sensitive film, the portion where a visual change is made). That is, the data of the two-dimensional positional information (e.g., X, Y) of the detection distribution of the pressure sensitive means including the occlusal pressure distribution information and the detection information (C_{XY}) are obtained.

Here, the pressure sensitive means is not particularly limited, and pressure sensitive films (pressure sensitive sheets), pressure sensitive sensors that detect the occlusal contact position by variations in electrical characteristic, and the like may be exemplified. Among them, pressure sensitive films are preferable from the viewpoint of higher versatility and easy display of occlusal pressure.

The method of obtaining the data of positional information and detection information from the pressure sensitive means is not particularly limited. For example, if the pressure sensitive means is a pressure sensitive film, scanners and cameras for reading color or shade appeared on the pressure sensitive film may be used. If the pressure sensitive means is a pressure sensitive sensor, devices for measuring electrical features (voltage, current, resistance value, and changes in these features) may be exemplified.

As a more specific example, for each minimum unit (for example, one pixel), the coordinates thereof may be used as the positional information, and the color (RGB value) may be used as the detection information.

### Step S12 of Calculating Pressure Value

In the step S12 of calculating pressure value (it may be referred to as “step S12”), the detection information obtained in the step S11 is converted into a pressure value. That is, for each minimum unit, the obtained detection information (RGB value in the case of a pressure sensitive film) is converted into a pressure value by a relational expression obtained in advance. This provides a pressure value for each minimum unit.

### Step S13 of Determining Detection Unit and Detection Group

In the step S13 of determining detection units and detection groups (it may be referred to as “step S13”), a positron where the pressure value is obtained and a position where the pressure value is not obtained are sorted out from the pressure value obtained in step S12 (identification of the detection units), and for a portion in which a plurality of detection units form a continuous one mass, its area is obtained as one detection group, and a characteristic value based on the pressure is calculated.

From this, a plurality of detection groups and detection units that does not belong to the detection groups are determined, and for each of them, the characteristic value based on the area and the pressure is obtained.

More specifically, for example, the following processes are performed.

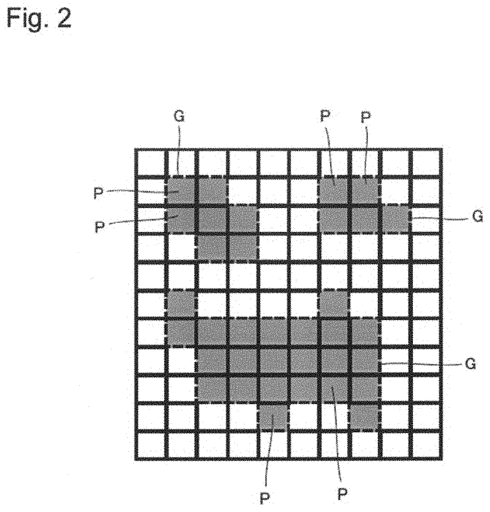

As shown in FIG. 2, the minimum unit where the pressure value is obtained (the pressure is not 0) for each minimum unit (pixel in this embodiment) is used as the detection unit. In FIG. 2, each pixel filled with thin gray is a detection unit P. When some of the detection units among the plurality of detection units are adjacent to each other and exist continuously, the detection units adjacent to each other are considered as one detection group. Each of the areas surrounded by dashed lines in FIG. 2 is a detection group G.

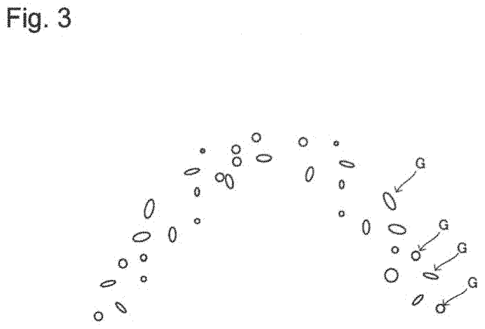

Specifically, a plurality of detection units, which exist continuously and form a group, are defined as the detection group by labeling. In this embodiment, the 8-connectivity processing is used to make the detection units continuous in the vertical, horizontal, and diagonal directions into the same label. This makes it possible to obtain distributions of the detection groups having different areas, as schematically shown in FIG. 3.

For each detection group, the area of the detection group is calculated from the number of the detection units belonging to the detection group, and the pressure characteristic value is calculated based on each pressure value of the detection units belonging to the detection group. This pressure characteristic value is a pressure value to characterize the pressure state of the detection group and to treat the detection group as one pressure state. Specifically, for example, the average value of the pressures of the detection units belonging to the detection group can be set as the pressure characteristic value, or the maximum value or the minimum value of the pressure value of the detection units belonging to the detection group can be set as the pressure characteristic value.

For a detection unit that docs not belong to any detection groups and exists independently of other detection units, its pressure value is used as the pressure characteristic value.

### Separation Step S14 with Threshold

In the step S14 (it may be referred to as “step S14”), the detection groups and the detection units (not belonging to any detection groups) are separated based on the pressure characteristic value into the ones that can be regarded as detection due to occlusion and the ones that are determined to be unclear as to whether the detection is due to occlusion.

If the pressure characteristic value is equal to or larger than the predetermined pressure characteristic value, the detection groups and the detection units (not belonging to any detection groups) can be determined that they can be regarded as due to occlusion and not due to noise without taking other matters into consideration. Therefore, the detection groups and the detection unis (not belonging to any detection groups) whose pressure characteristic value is equal to or larger than the predetermined pressure characteristic value are regarded as due to occlusion. Specifically, for example, the determination includes “the detection groups and the detection units (not belonging to any detection groups) whose pressure characteristic value is equal to or greater than P₁ [MPa] are the detection unit due to occlusion (P₁ is a specific numerical value)”.

### Separation Step S15 with Consideration of Area

In the separation step S15 (it may be referred to as “step S15”) with the consideration of the area, the detection groups and the detection units (not belonging to any detection groups) which are determined to be due to occlusion in the step S14 (not belonging to any detection groups) are further separated into the detection groups and the detection units (not belonging to any detection groups) which are determined to be due to occlusion and the detection groups and the detection units (not belonging to any detection groups) which are unclear whether they are due to occlusion, based on the threshold of the pressure characteristic value associated with the area.

In step S14, even among the detection groups and the detection units (not belonging to any detection groups) which are unclear whether the detection is due to occlusion, the separation is made into the detection groups and the detection units (not belonging to any detection groups) which are considered to be based on occlusal contact and the detection groups and the detection units which are unclear whether the detection is due to occlusion, from the data base in which the relationship between the area of the occlusal contact portion and the occlusal pressure thereof has been searched in advance.

With this selection, the occlusal contact state can be represented more accurately.

For example, with respect to the detection groups and the detection units (not belonging to any detection groups) which are determined in S14 as unclear whether the detection is due to occlusion, if the area is equal to or larger than A₁ mm² and smaller than A₂ mm², it is determined that it is all unclear whether the detection is due to occlusion (for example, making P₁ [MPa] as the upper limit of the threshold, all the detection groups and the detection units whose pressure characteristic value is less than P₁ [MPa]), and with respect to the detection groups and the detection units (not belonging to any detection groups) whose area is equal to or larger than A₂ mm², making P₂ [MPa] as the upper limit of the threshold, the detection groups and the detection units (not belonging to any detection groups) having a pressure characteristic value higher than P₂ [MPa] are considered to be due to occlusion. Here, each of A₁, A₂, P₂ is a specific number (P₁ is the same as above). However, A₁>A₂ and P₁>P₂. Specific numerical values entering there can be determined from the characteristics of the pressure sensitive means (pressure sensitive sheet, etc.), and the values are obtained in advance depending on the pressure sensitive means to be used. The method for obtaining the specific numerical values is not particularly limited, and if the pressure sensitive means is a pressure sensitive sheet, it is possible, for example, to intentionally generate the detection units and the detection groups by giving an assumed bending or slippage to the pressure sensitive sheet, and the value generated by tins may be used.

The relationship between the areas of the detection groups and the detection units not belonging to any detection groups and the threshold of the upper limit of the pressure characteristic value is not particularly limited, and the upper limit of the pressure characteristic value at the threshold can be made to be smaller as the areas of the detection group and the detection unit not belonging to any detection groups is larger. This can further improve the accuracy.

### Other Separation Step S16

In addition to the above, in the other separation step S16 (it may be referred to as “step S16”), when the detection group is a line segment, a separation may be carried out at least by one of: the detection groups and the detection units (not belonging to any detection groups) which are clearly separated from the adjacent detection groups or the detection units (not belonging to any detection groups); and the color of the detection groups and the detection units not belonging to any detection groups.

With respect to the color of the detection groups and the detection units not belonging to any of the detection groups, for example, it is possible to form a database in advance based on R value, G value, and B value in associate with that the detection is not due to occlusion when the color does not fall in a predetermined range. However, this step S16 is not necessarily provided, and may be provided as necessary.

### Notification Step S17

In the notification step S17 (it may be referred to as “step S17”), the detection groups and the detection units (not belonging to any detection groups) regarded as due to occlusion by the various separations in the steps S14 to S16, and the ones whose detection is unclear whether it is due to occlusion, are notified differently. The method of notification is not particularly limited, and may be performed by, for example, color, brightness, and both on a screen.

How to treat the detection groups and the detection units (not belonging to any detection groups) which are notified in the step S17 and whose detection is unclear whether it is due to occlusion may be appropriately set as necessary. For example, it may be specified and deleted one by one, or to some extent collectively, all notified detection groups and detection units (not belonging to any detection groups) may be specified collectively and automatically deleted, and may be manually changed to detection groups and detection units (not belonging to any detection groups) due to occlusion.

According to the occlusal pressure analysis program S10 of the present embodiment, in obtaining the occlusal pressure by the pressure sensitive means, it is possible to separate a portion due to occlusion and a portion not due to occlusion accurately and efficiently, and it is possible to improve the accuracy of grasping the occlusal contact state.

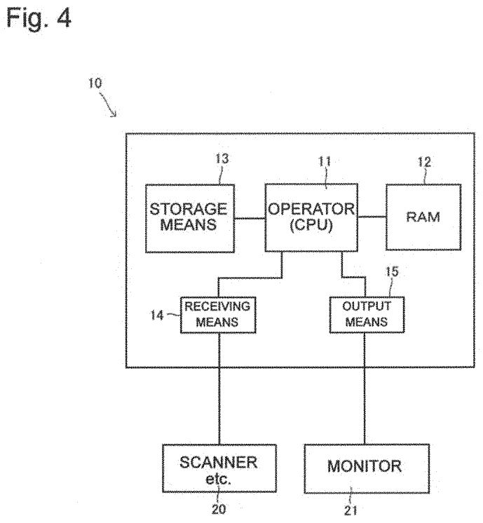

The occlusal pressure analysis program S10 to which these steps belong is performed by an electronic computer such as a computer. That is, the occlusal pressure analysis program S10 including the steps S11 to S17 is performed by an electronic computer performing an operation and outputting the results. FIG. 4 shows a diagram for explanation. As shown in FIG. 4, an electronic computer 10 includes an operator 11, a RAM 12, a storage means 13, a receiving means 14, and an outputting means 15.

The operator 11 is composed of a so-called CPU (Central Processing Unit), and is connected to the above-mentioned structural members and is a means capable of controlling them. The operator 11 also executes various programs stored in the storage means 13 or the like that functions as a storage medium, and performs an operation as a means for generating various data and selecting data based on the programs. In this embodiment, the storage means 13 stores live occlusal pressure analysis program S10 and the operator 11 operates the program to obtain the results.

RAM 12 is a member that functions as a working area of the operator 11 and a temporary data storage means. RAM 12 may be composed of a SRAM, DRAM, a flash memory, or the like, and is similar to known RAMs.

The storage means 13 is a member that functions as a storage medium for storing programs and data as a basis for various operations. The storage means 13 may also be capable of storing intermediate and final results obtained by the execution of the program. In this embodiment, the occlusal pressure analysis program S10 is stored in the storage means 13.

The receiving means 14 is connected to a scanner 20 for example, and is a member having a function to incorporate image information based on the pressure sensitive means from the scanner.

The output means 15 is a member having a function to output information to be output to the outside among the results obtained by the operation of the operator 11, and in this embodiment, is connected to the monitor 21, from which the person who operates the electronic computer can see the result on the screen.

According to the electronic computer 10, the data presented on the pressure sensitive means from a reading medium such as a scanner is taken into the electronic computer 10 via the receiving means 14, the operator 11 performs an operation based on the occlusal pressure analysis program S10 stored in the storage means 13, and the results are displayed on the monitor 21 via the output means 15.

This makes it possible for the person who operates the computer to obtain the occlusal contact state in which data of an unnecessary portion is finally deleted and accuracy is enhanced.

### REFERENCES SIGN LIST

10 electronic computer
11 operator
12 RAM
13 storage means
14 receiving means
15 output means

1. An occlusal pressure analysis program for obtaining an occlusal pressure distribution from a pressure sensitive means, the occlusal pressure analysis program comprising:
a step of obtaining a pressure value for each minimum unit from which the pressure value can be recognized by the pressure sensitive means; and
a step of, regarding each of the minimum units where the pressure value exists as a detection unit, separating the detection units into the detection units which can be regarded as due to occlusion and the detection units which are unclear whether they are due to occlusion, based on the pressure value.

2. An occlusal pressure analysis program for obtaining an occlusal pressure distribution from a pressure sensitive means, the occlusal pressure analysis program comprising:
a step of obtaining a pressure value for each minimum unit from which the pressure value can be recognized by the pressure sensitive means;
a step of obtaining areas of detection groups, regarding the minimum unit where the pressure value exists as a detection unit and regarding a group of a plurality of the detection units that are existing continuously as each of the detection groups;
a step of obtaining a pressure characteristic value of each detection group; and
a step of separating the detection groups into the detection groups which can be regarded as due to occlusion and the detection groups which are unclear whether they are due to occlusion, based on the area of the detection group and the pressure characteristic value.

3. The occlusal pressure analysis program according to claim 2, comprising a step of separating the detection groups into the detection groups which can be regarded as due to occlusion and the detection groups which are unclear whether they are due to occlusion, by a threshold of the pressure characteristic value associated with the area of the detection group.

4. The occlusal pressure analysis program according to claim 3, wherein an upper limit of the pressure characteristic value at the threshold becomes smaller as the area of the detection group is larger.

5. The occlusal pressure analysis program according to claim 2, further comprising a step of separating the detection groups into the detection groups which are due to occlusion and the detection groups which are unclear whether they are due to occlusion, from at least one of the shape, distance from the other detection groups, and color of the detection group.

6. The occlusal pressure analysis program according to claim 3, further comprising a step of separating the detection groups into the detection groups which are due to occlusion and the detection groups which are unclear whether they are due to occlusion, from at least one of the shape, distance from the other detection groups, and color of the detection group.

7. The occlusal pressure analysis program according to claim 4, further comprising a step of separating the detection groups into the detection groups which are due to occlusion and the detection groups which are unclear whether they are due to occlusion, from at least one of the shape, distance from the other detection groups, and color of the detection group.
